(19) United States
(12) Patent Application Publication
(10) Pub. No.: US 2017/0369167 A1
(43) Pub. Date: Dec. 28, 2017

(54) PRECISE AND RAPID DELIVERY OF AN EMERGENCY MEDICAL KIT FROM AN UNMANNED AERIAL VEHICLE

(71) Applicant: 1st Rescue, Inc., Jacksonville Beach, FL (US)

(72) Inventor: William D. Meadow, Jacksonville, FL (US)

(73) Assignee: 1st Rescue, Inc., Jacksonville Beach, FL (US)

(21) Appl. No.: 15/631,744

(22) Filed: Jun. 23, 2017

Related U.S. Application Data
(60) Provisional application No. 62/354,169, filed on Jun. 24, 2016.
(60) Provisional application No. 62/363,805, filed on Jul. 18, 2016.

(51) Int. Cl. B64D 1/02; G05D 1/00; B64C 39/02; A61N 1/39

(52) U.S. Cl. CPC B64D 1/02; B64C 39/024; G05D 1/0038; A61N 1/39; B64C 2201/146; B64C 2201/128

(57) ABSTRACT

Embodiments relate to methods and apparatus to swiftly and accurately deliver a medical treatment kit via an Unmanned Aerial Vehicle and mechanisms and systems for monitoring a medical emergency via an Emergency Control Center and providing live remote support to on location care givers.

### CROSS-REFERENCE TO RELATED APPLICATIONS

This application claims the benefit of U.S. Provisional Patent Application Ser. No. 62/354,169 filed on Jun. 24, 2016 entitled METHOD AND APPARATUS FOR PRECISE AND CONTROLLED DELIVERY OF A PAYLOAD FROM UNMANNED AERIAL VEHICLES, and also U.S. Provisional Patent Application 62/363,805, filed Jul. 18, 2016 and entitled PRECISE AND RAPID DELIVERY OF AN EMERGENCY MEDICAL KIT FROM AN UNMANNED AERIAL VEHICLE the entire contents of each of which is hereby incorporated by reference in its entirety.

### FIELD OF THE INVENTION

Embodiments of the present invention include methods and apparatus for expedited delivery of a medical treatment kit (“MedKit”) to a site of a medical emergency via precise identification of a delivery site and controlled delivery of the medical treatment kit from an aerial vehicle to the site of the emergency. More specifically, an emergency radio communication may be received by an Emergency Command Center or Public Service Access Point, which will dispatch a drone with a medical treatment kit to while an emergency response team is in route to the scene of the emergency.

### BACKGROUND

As early as the 1950s the use and development of unmanned aerial vehicles (“UAVs”), also known as drones, has taken place. Since then, early and in some aspects current development efforts have been limited mainly to military applications, hindering the discovery, use, and developments of Unmanned Aerial Vehicles for some applications. More recently, other elements associated with the use of Unmanned Aerial Vehicles including control systems, control linked databases, support equipment, etc., beyond the Unmanned Aerial Vehicle itself has been explored to enable the use of Unmanned Aerial Vehicles not only in military applications but also to provide utility in other applications.

As the use of different types of aerial vehicles rapidly expands in both military and more recently non-military applications, different retrieval systems, refueling systems, guidance systems, and operating systems capable of allowing communication between multiple aerial vehicles and databases have been described. Such systems are intended control and project flight patterns and warning using other identified aircrafts or approaching objects. Recently and more specifically related to the subject matter of this application, a variety of methods describing recognition patterns and landing adaptations for aircrafts have been described in the following references.

Some known systems or methods of the background art describe a ground marker for the orientation and sign navigation of a drone. Such background art describes a marker with certain shape and color pattern characteristics that can be placed on the ground and be remotely recognized by a drone through low on-site incidence according to predefined asymmetrical geometries.

Some known systems or methods of the background art describe an unmanned aerial vehicle control system. Such background art describes a method switching flight control modes of an aircraft upon landing or takeoff. The flight control modes varying from autonomous modes and manual modes based on the indication of landing or takeoff.

Some known systems or methods of the background art describe a method for vertical takeoff and landing on inclined surfaces. Such background art describes a method for the vertical landing and taking off of an aerial vehicle on an inclined surface based on a measured pressure differential and data from multiple sensors describing contact from various contact points of the unmanned aircraft with the inclined surface. The pressure differential and data from the multiple sensors is used to adjust vanes and the thrust of the unmanned aerial vehicle.

However, in the descriptions of the aforementioned background art, no reference has described, enabled, or provided motivation to combine the described developments with another or the methods presented herein to enable improved delivery of an interactive video and data medical treatment kit transported by an unmanned aerial vehicle.

As a consequence of the foregoing, new methods and apparatus are desired that can provide a practical method and apparatus for the identification and control of Delivery Zones for various aerial vehicles. The desired novel method and apparatus being the subject matter of embodiments of the present invention described in succeeding sections of this application.

### SUMMARY OF THE INVENTION

Accordingly, embodiments of the present invention include methods and apparatus for identification of a site of a medical emergency and delivery of a medical treatment kit to a delivery zone identified that is proximate to the site of the medical emergency and delivery of video and bio-telemetry data from the medical treatment kit to the synchronously deployment of an emergency response team to the site of the medical emergency. A drone or other UAV delivers the medical treatment kit via direct aerial access and may implement controlled placement of the medical treatment kit in the Delivery Zone.

More specifically, identification of a Delivery Zone for a particular medical treatment kit carried by an Unmanned Aerial Vehicle may be based upon a geospatial coordinate determined from geocoder look up of a street address or a verbal description of a point of interest or locally known landmarks to an emergency response operator viewing a map that supports geocoding, with numeric values based on such as a global positioning system (GPS) coordinate and image capture verification. Image capture verification may be accomplished for example via a camera carried by the Unmanned Aerial Vehicle which captures image data of an approaching Delivery Zone and transmits imagery in real time to a remote operator. One or both of a user and a database of previously stored image data may visually verify that the Delivery Zone is a proper deliver zone.

Controlled placement of the medical treatment kit carried by the Unmanned Aerial Vehicle may be accomplished via a Delivery Zone Drop Line that is extended from the Unmanned Aerial Vehicle to the Delivery Zone. In some simple embodiments, the Delivery Zone Drop Line will be dropped with the payload medical treatment kit secured to the bottom of the Delivery Zone Drop Line, in which case the delivered medical treatment kit is lowered into the Delivery Zone while the UAV hovers overhead providing a higher altitude tower to relay radio transmissions from the medical treatment kit or its contents on the ground below to the radio transmitter on the UAV.

In other embodiments, a Delivery Zone Drop Line may be lowered with a horizontal positioning unit, such as a thruster secured near the bottom of the Drop Line. In one embodiment, the thruster positions the Drop Line so the medical treatment kit is precisely over the Delivery Point, for the medical treatment kit to be lowered to surface of the Delivery Zone.

In some embodiments, a registration database of image data of Delivery Zones may be used to classify and provide information pertaining to a particular Delivery Zone and its surroundings. In some embodiments of the present invention, the registration database may include profiles that optionally can be remotely updated via image recognition of features in the Delivery Zone and/or indicator symbols seen by a remote operator of the UAV, the symbols providing essential landing information that may pertain to one or more of the surroundings of the Delivery Zone, its geometry, capacity, accessibility, and delivery feature information for picking up and delivery of a medical treatment kit transported by the Unmanned Aerial Vehicle.

In other embodiments of the present invention, three dimensional (3-D) geometries or point cloud around landings may be used for identification and subsequent registration of a Delivery Zone in the database or for verification of database transmitted information. For example, an electronic signal may be received by a sensor on the aerial vehicle, or an imaging system may superimpose three dimensional point cloud data around a Delivery Zone with one or more captured images to verify or register a particular Delivery Zone.

Information about registered Delivery Zones may include, for example, weather or wind information, flight restriction data, security codes (e.g., answers to questions from governmental air traffic control organizations, protocols for being granted flight and landing rights), security status (whether the UAV is allowed to land here at this time), and security quality (e.g., what level of authentication is required to request landing rights and or open access doors to gain entry to touchdown area). Moreover, related to the location where the UAV is trying to land, information may include surrounding information locations for additional storage of supplies, products, tools, batteries, fuel, fuel cells, or merchants, etc.

Delivery Zones may be equipped with additional support features to receive, deliver or store a medical treatment kit. For example, support features may include a temperature controlled compartment, a security system to keep a medical treatment kit secure, a weight station to confirm the contents of a medical treatment kit, a tarp or the like comprising a protective cover to prevent damage of the medical treatment kit due to weather conditions.

Delivery Zones for the system may include modes of communication linked to databases and or processor included in the aerial vehicle. Consequently, in some embodiments a Delivery Zone is not required to be stationary or fixed upon a structure but rather may be transported by another aerial vehicle or be a mobile registration delivery zone. For example, an Unmanned Aerial Vehicle may recognize a porch in a single or balcony of a multi story building associated with a person or address as a proper Delivery Zone.

Delivery Zone registration may be accomplished via a user logging into a system, such as a webpage or an application in a smart phone, and registering a location and general features using the smart phone's GPS and camera to provide image data information. Further, the Delivery Zone may be defined by a visual indicator that could be a standard symbol or it could also include a unique value such as a bar code or radio transmitter as a uniquely identified site specific beacon that is placed upon a structure that provides information about the Delivery Zone. The surface on which the Delivery Zone can be a surface including, grass, glass, a solar panel capable of recharging the aerial vehicle, a concrete balcony, etc. provided that information relevant to the particular surface can be linked to it. For example, in a glass surface weight limitations can be registered in the system. Another example includes a Delivery Zone associated with a concrete balcony. In such embodiments, an altitude and clearance dimensions and approach information may be registered into the system either automatically or manually.

One general aspect includes a system to control placement of a payload carried by an unmanned aerial vehicle (UAV) at a delivery zone, the system including: a payload coupled to the UAV. The system also includes a location determination device to determine a location of the UAV. The system also includes a drop line including a proximate end coupled to the UAV and a distal end for locating in a delivery zone. The system also includes a descent regulator to control a rate of descent of the payload from the UAV to the distal end of the drop line at the delivery zone via descent of the payload along the drop line. The system also includes a release mechanism to release the payload from the drop line. Other embodiments of this aspect include corresponding computer systems, apparatus, and computer programs recorded on one or more computer storage devices, each configured to perform the actions of the methods.

Implementations may also include one or more of the following features. The system may further include a position control unit to control a position of the UAV in a horizontal plane. The system may include examples where the position control unit further controls one or both of: an altitude of the UAV and a height of the UAV. In some examples, the system further includes an altitude control unit to control one or both of an altitude of the UAV and a height of the UAV. The system may further include an operator interface to control navigation of the UAV based upon instructions transmitted to the position control unit. The system may further include a thruster coupled to the payload, the thruster to provide more accurate positioning of the payload in a lateral direction as compared to accuracy of lateral positioning enabled by the position control unit.

The system may further include an operator interface to control positioning of the thruster. The system may include examples where the delivery zone is described by image data and is recognized by the UAV by use of image recognition. The system may include examples where the delivery zone includes a mobile target. In some examples, the delivery zone includes a temporary target described by image data, and the temporary target is recognized by the UAV by use of image recognition. The system may include examples where the delivery zone is marked by a radio transmitter. In some examples, the payload includes a medical treatment kit. In some examples, the medical treatment kit includes a heart defibrillator.

The system may also include examples where the medical treatment kit includes a voice communication link with an emergency command center. The method may further include a step of, prior to releasing the payload, controlling a position of the payload separately from control of a position of the UAV. The method may further include a step of providing a voice communication link from the payload to an emergency command center. The method may further include a step of identifying the delivery zone by recognition of visible features. The method may further include a step of identifying the delivery zone by identification of non-visible features. Implementations of the described techniques may include hardware, a method or process, or computer software on a computer-accessible medium.

One general aspect includes a system providing precise placement of a payload carried by an unmanned aerial vehicle (UAV) to a delivery zone, the system including: a mechanism to fixedly attach a payload to the UAV. The system also includes a location determination device to determine a location of the UAV. The system also includes a drop line including a proximate end coupled to the UAV and a distal end for positioning in a delivery zone. The system also includes a coupling device for slidingly attaching the payload to the drop line. The system also includes a descent regulator to control a rate of descent of the payload from the UAV to the delivery zone via the drop line. The system also includes a release mechanism to release the payload at the delivery zone. The system also includes an electronic control unit for controlling a location of the UAV. The system also includes a height controller for controlling a height of the UAV. The system also includes a thruster unit for controlling x and y movement of the payload while the payload is suspended from the drop line. The system also includes a delivery zone determination unit for determining a location for delivery within a one meter accuracy. The system also includes a delivery zone coupler for coupling the thruster unit to the delivery zone during delivery. Other embodiments of this aspect include corresponding computer systems, apparatus, and computer programs recorded on one or more computer storage devices, each configured to perform the actions of the methods.

One general aspect includes a method to control placement of an item carried by an unmanned aerial vehicle (UAV) at a delivery zone, the method including: coupling a payload to the UAV. The method also includes determining a location of the UAV by use of a location determination device. The method also includes lowering the payload at the delivery zone, at a controlled rate of descent, by use of a drop line. The method also includes releasing the payload at the delivery zone.

### BRIEF DESCRIPTION OF THE DRAWINGS

The accompanying drawings, that are incorporated in and constitute a part of this specification, illustrate several embodiments of the invention and, together with the description, serve to explain the principles of the invention:

FIG. 1 illustrates delivery of a medical treatment kit to a delivery zone via a UAV, in accordance with an embodiment of the present invention;

FIG. 2A illustrates preparation of delivery of a medical treatment kit to a precise location via a UAV in accordance with an embodiment of the present invention;

FIG. 2B illustrates delivery of a medical treatment kit to a precise location via a UAV in accordance with an embodiment of the present invention;

FIG. 3A illustrates a UAV lowering a medical treatment kit without a thruster in accordance with an embodiment of the present invention;

FIG. 3B illustrates a UAV lowering a medical treatment kit with a thruster in accordance with an embodiment of the present invention;

FIG. 4 illustrates a usage scenario in accordance with an embodiment of the present invention; and

FIG. 5 illustrates a medical treatment kit in accordance with an embodiment of the present invention.

### DETAILED DESCRIPTION

Embodiments of the present invention provides specialized apparatus and methods of operating the specialized apparatus, in order to identify a Delivery Zone and to deliver precisely a shipped medical treatment kit into the Delivery Zone in a controllable manner. A controllable manner may include for example deliberate and careful placement of the shipped medical treatment kit into the Delivery Zone with a reduced force of impact as compared to dropping the medical treatment kit from the Unmanned Aerial Vehicle or requiring the UAV to land and then release the Payload.

In the above sections, detailed descriptions of embodiments of the invention will be given. The description of both preferred and alternative embodiments though through are exemplary only, and it is understood that to those skilled in the art that variations, modifications and alterations may be apparent. It is therefore to be understood that the exemplary embodiments do not limit the broadness of the aspects of embodiments of the underlying invention as defined by the claims.

A Delivery Zone may include, for example, X, Y and Z coordinates, wherein the X and Y coordinates designate a geospatial coordinate in a horizontal plane, and the Z coordinate designates a vertical altitude or height. Height may be a vertical distance with respect to a distance above a surface of the Earth at the X, Y location, and altitude may be a vertical distance with respect to sea level.

Glossary:

“Control Reel” as used herein refers to a mechanism to dispense and/or collect a Delivery Line from an UAV to the medical treatment kit.

“Delivery Point” refers to a point within the Delivery Zone designated as a target place to receive a payload medical treatment kit.

“Delivery Line” refers to a pliable line, such as a monofilament line, cable, rope, belt or other reel-able extension vehicle.

“Delivery Zone” refers to a designated area for delivery of a payload medical treatment kit.

“Payload” or “Payload Medical Treatment Kit” refers to an article carried by a UAV to be delivered according to the methods and apparatus described herein. Exemplary payloads may include, but not limited to, an automated external defibrillator, a wearable device or a sticker based bio-telemetry devices (e.g., to monitor heart rate, blood pressure, EKG, pulse oximetry, etc.), quick clot bandages, cold cap, tourniquets, injectables (e.g., naloxone hydrochloride known by the trade name Narcan®, an epinephrine auto-injector known by the trade name EpiPen®, etc.), first aid kit, camera, loud speaker, a hat with wireless video camera attached, Wi-Fi transmitter, computer tablet and power supply. Usage of certain medical devices such as a defibrillator may be extremely time-sensitive, and fast access to such devices is critical if they are not already onsite.

“Thruster” refers to a device attachable to a Delivery Line that is capable of X, Y positioning of a distal end of the Delivery Line while the proximate end of a Delivery Line is attached to an UAV. The thruster may include a secondary UAV that is capable of at least X, Y movement, while Z movement is optional since the Z movement may be provided by the Unmanned Aerial Vehicle carrying the Payload to be delivered.

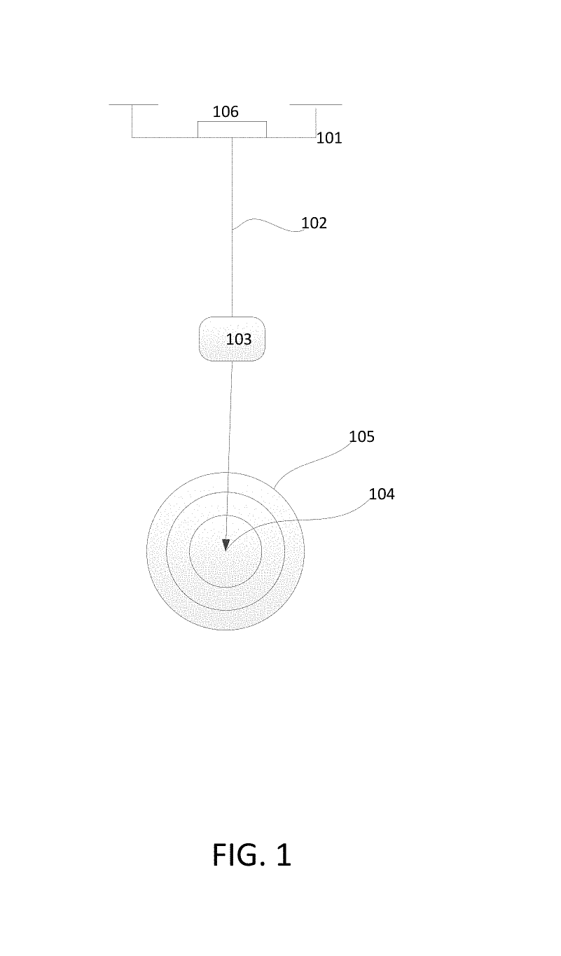

Referring now to FIG. 1, an Unmanned Aerial Vehicle 101 is shown with a Delivery Line 102 extended from the Unmanned Aerial Vehicle towards a Delivery Zone 105. The Delivery Zone 105 also may include a Delivery Point 104 that includes the preferred point of delivery. The Unmanned Aerial Vehicle 101 is positioned to a place in the general proximity of the Delivery Zone 105, e.g., a place within range of Delivery Line 102. Once in the general proximity, UAV 101 may hover or linger above (but not necessarily directly above) Delivery Zone 105. A navigation unit (e.g., a global positioning system (GPS) receiver, a beacon receiver, an inertial guidance or dead reckoning unit, image matching analyzer, etc.) may guide or position the Unmanned Aerial Vehicle 101 via geospatial coordinates, and also may include a vertical coordinate in case Delivery Point 104 is not at ground level. The Delivery Line 102 may be extended from the Unmanned Aerial Vehicle 101 towards the Delivery Zone 105, and a Payload Item which in some examples is a medical treatment kit 103 extendibly coupled to UAV 101 by Delivery Line 102 may be delivered onto the Delivery Zone 105. In some embodiments, a descent regulator (e.g., a friction clamp, a brake, a clutch, a motor control, a motor holding torque, or the like) may be used to further manage a rate of descent of a payload medical treatment kit from the UAV 101 via the Delivery Line 102.

In preferred embodiments, the Delivery Line 102 may include a light weight flexible line, such as a monofilament line, and be lowered via an automated reel 106 driven by a motor (e.g., a stepper motor, a servo motor, or other mechanism). The Delivery Line 102 may be selected according to the lift weight of the Unmanned Aerial Vehicle 101 and the weight of a medical treatment kit 103 to be delivered. Some embodiments, for general use of delivering packages may include an eighty-pound test line, although other strengths, such as a line between 12 lbs. and hundreds of pounds test also may be used in various embodiments. In some additional embodiments, other types of line, such as a carbon nano line, or a steel multi-strand cable also may be used as a Delivery Line 102.

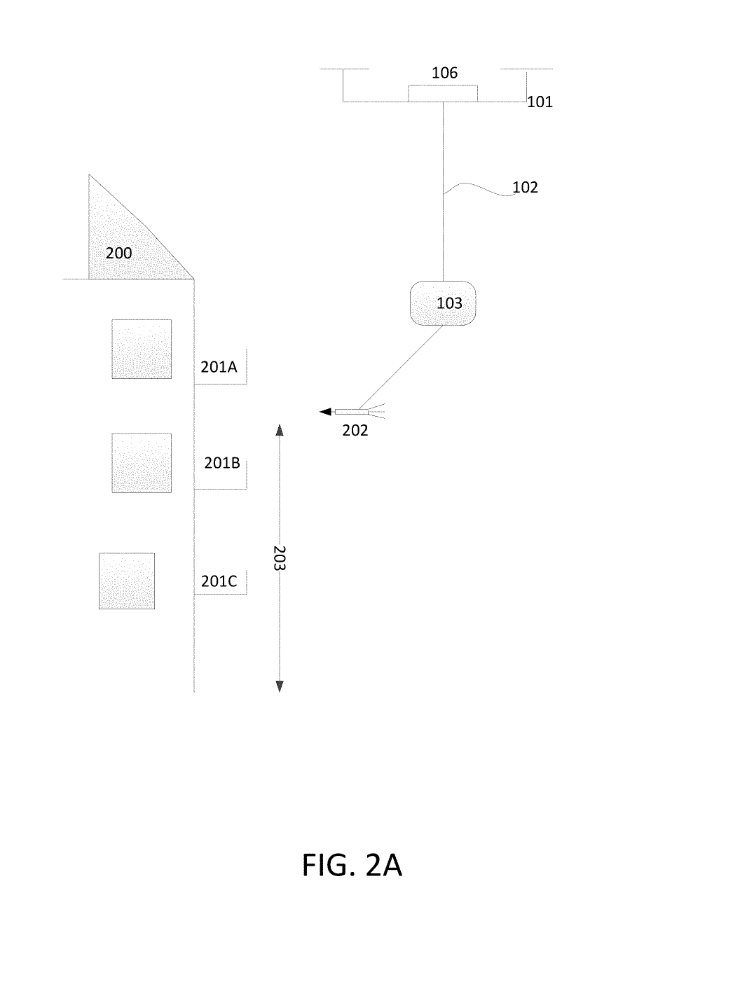

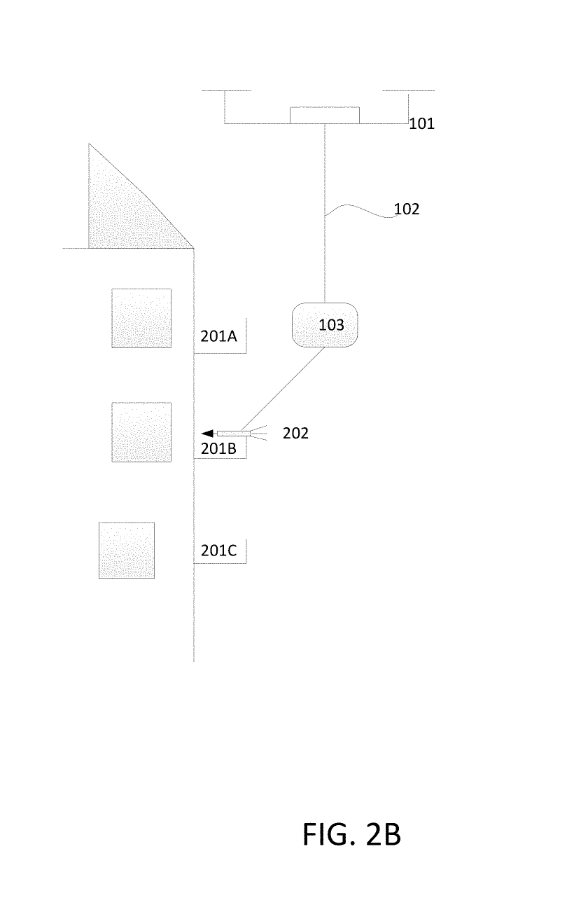

Referring now to FIG. 2A, in some embodiments, a Delivery Zone may include a structure 200 or land formation (e.g., a cliff face) with a vertical dimension 203. In addition, in some embodiments an Unmanned Aerial Vehicle 101 may not be able to easily access a point directly vertical to a Delivery Zone 201A-201C for a straight down drop of a Payload of a medical treatment kit 103. For example there may be difficult to see obstructions such as power lines as well as tree branches that have grown toward the open areas where the risk of wind causing an impact on decent to land is too high. In these, and all other embodiments, a thruster 202 may be attached to the Delivery Line 102 and used to finely position the Delivery Line 102 to a correct position for delivery of a medical treatment kit 103. Slack in Delivery Line 102, swinging of Delivery Line 102, wind, and similar effects may make it very difficult to achieve accurate positioning of the Payload medical treatment kit 103 without use of thruster 202. A remote operator may navigate the UAV 101 and place the UAV 101 in a hover mode where its XYZ position is automatically maintained while the operator controls the X, Y fine positioning of the thruster 202. The thruster 202 may include, e.g., a fan, a mini Unmanned Aerial Vehicle, or other device capable of at least X, Y movement. Z movement (within limits imposed by Delivery Line 102) is optional since the Z movement may be provided by the Unmanned Aerial Vehicle 101 carrying the medical treatment kit 103 to be delivered. Thruster 202 and/or UAV 101 may include a stabilizer (e.g., a gyroscope) to help compensate for position variations such as may be caused by wind.

Referring now to FIG. 2B, the thruster 202 is shown positioned in the desired Delivery Zone 201B. Once the thruster 202 is properly positioned, it may be secured via a Delivery Point securing device, or thruster 202 may be fixedly attached by a person or attendant (e.g., a nurse) who will receive medical treatment kit 103. Once thruster 202 is secured, medical treatment kit 103 may be lowered for delivery. A Delivery Point securing device may include, by way of nonlimiting example, a clasp, a clamp, a suction cup, instant set glue or sticky material, a hook and ring, a magnetic connection or other device to secure the Delivery Line 102 or thruster 202 to the Delivery Point.

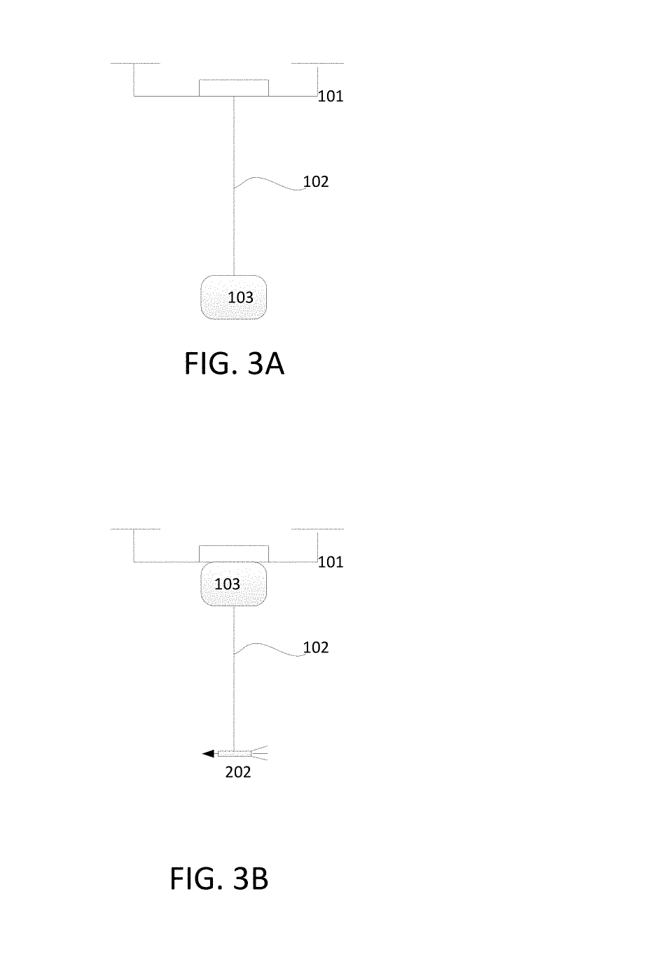

Referring now to FIG. 3A, an Unmanned Aerial Vehicle 101 is shown with a Delivery Line 102 and a medical treatment kit 103 being lowered without a thruster 202. FIG. 3B includes a Delivery Line 102 with a thruster 202 while the package is secured to the Unmanned Aerial Vehicle 101. The medical treatment kit 103 may stay secured to UAV 101 until the Delivery Line 102 is secured to a Delivery Point.

Functions and method steps may be locally controlled via a processor and software within the UAV 101. Alternatively, functions and methods may be remotely controlled via software and processors remote to the UAV 101 and in digital communication with the Unmanned Aerial Vehicle 101 via a wireless communications network. A combination of remote control and local control also may be used, e.g., local control to control flight dynamics of UAV 101 to provide a hover mode, and remote control to control whether to enter or exit hover mode. Image data relating to one or both of the Delivery Zone and the Delivery Point may be collected by the Unmanned Aerial Vehicle 101 or be transmitted to the UAV 101 via the communications network.

In another aspect, as a way to establish one or more Delivery Zones, users may login and register their location using a mapping application on their smart phone or PC. The mapping application may designate a location that is accurate within a few meters, typically from satellite data (e.g., GPS or satellite imagery). In some embodiments, a user also may log in and draw a designated set of characters (which can provide authentication for the Unmanned Aerial Vehicle 101) that is easy to recognize, and that also may designate a precise spot that the user wants the Unmanned Aerial Vehicle 101 to land in the future. The landing spot may include various types of surfaces, such as, for example, one or more of: grass, concrete, tarmac, wood, composite, glass, plastic, mesh, or other support structure capable of supporting the Unmanned Aerial Vehicle 101.

In another aspect, the Delivery Zone 105 and/or the Landing Zone may be described by image data, and UAV 101 may recognize the Delivery Zone 105 and/or Landing Zone by use of image recognition. For example, when an Unmanned Aerial Vehicle 101 approaches a Landing, one or more of the Unmanned Aerial Vehicle 101, the Landing and an electronic support module may generate a 3-D model confirming that the Unmanned Aerial Vehicle 101 is within the spatial clearance guidelines needed for the Unmanned Aerial Vehicle 101 to finish landing. As the Unmanned Aerial Vehicle 101 approaches the Landing, the system may confirm the landing based upon visual information and a model developed from a 3-D model dynamically in the landings. The Unmanned Aerial Vehicle 101 can then make a 360 video at the landing spot, which may be processed to build a 3-D data set that then may be transmitted to a landing controller or operator to confirm viability from high-resolution imagery.

A Delivery Zone marker may be a simple marker (e.g., a handwritten marker) that includes a special predetermined code for authentication. The code may have been generated by a control system and provided to a requestor when the UAV 101 was requested. An objective of the code is that the Unmanned Aerial Vehicle 101 using a camera or other detector should be able to recognize the code easily even with coarse resolution, such that an Unmanned Aerial Vehicle 101 with HD camera can detect and perform pattern recognition as far away as several hundred feet.

With respect to the mobile landings, once the delivery point is registered, the registration can be updated in real-time from a third-party data source such as GPS (e.g., by use of a smartphone app) that lets the Unmanned Aerial Vehicle 101 know and transmit to a cloud server a precise location of a Delivery Zone or an individual who needs delivery of a medical kit or other package. Delivery Zones may include a set of standard symbols that may be created to provide special navigational directions and indicators to the Unmanned Aerial Vehicle 101 in case there is a loss of communication and the Unmanned Aerial Vehicle 101 en route to a destination cannot obtain local navigation information. In case of communication breakdown (e.g., in a disaster zone), symbols may be laid out for an Unmanned Aerial Vehicle 101, to help it effectively land and be aware of hazards at a landing zone or delivery zone. Embodiments may be able to recognize special symbols and platforms that identify, warn of, or provide directions to avoid spatial constraints in particular areas.

For mobile Delivery Zones, such as moving Delivery Zones with people and delivery trucks etc., the Unmanned Aerial Vehicle 101 may integrate multiple data sets using different types of technology such as visual (e.g., camera), acoustic, radar, and so forth to build a point cloud to assist in navigation for a precise final landing. Sensors may be provided as a single detector per type of technology, or multiple detectors per type of technology in order to provide binocular distance.

In some embodiments, a temporary Delivery Zone may be visibly indicated by a predetermined object, or a portable beacon (e.g., a strobe light) or a predetermined surface (e.g., a tarp that may be unrolled to reveal predetermined recognizable markings). The temporary Delivery Zone may be described by image data, and the temporary target is recognized by the UAV 101 by use of image recognition.

In some embodiments, a temporary Delivery Zone may be indicated non-visible methods, e.g., by use of a radio beacon, an RFID signal, other kind of radio transmitter, an audible signal (e.g., a chirp, a tone, or other predetermined audible signal), an infrared signal or signature, an ultrasonic signal, and so forth.

The Delivery Zones can include special patterns like a 1-D barcode, a 2-D barcode (e.g., QR code or PDF417) so that current information about the Delivery Zones can be determined by the marks and displayed on a display screen. Landings zones may be large in size, and in an area that may be very large and identifiable by a grid pattern, where the grid pattern may appear dynamically so that the Unmanned Aerial Vehicle 101 can see a spot that UAV 101 or its payload is designated to land under the precision placement of a thruster 202.

In some embodiments, a landing spot may be specified at a location along a conveyor system. Accordingly a moving Delivery Zine may provide information for the Unmanned Aerial Vehicle 101 to indicate an appropriate payload transfer point at a designated point along a moving conveyor.

Each landing that is registered in a Delivery Zone database can have the three point cloud data stored so that the Unmanned Aerial Vehicle 101 can use it as it gets close to the Delivery Zone. UAV 101 may perform onboard processing of the 3-D point cloud to compare and confirm that nothing has changed in the 3-D space of the Delivery Zone that would cause an additional safety risk (e.g., a previously unknown obstacle moved into the Delivery Zone).

Delivery Zones also may include moving walls that will open and shut to create a shell to provide cover from weather and secure access. The opening and closing of the shell can be based on appropriate security protocols. A secure database may be accessed from the Unmanned Aerial Vehicle 101 while landing, and can include challenge response codes in order to open the shell for access and to convey goods for delivery. If secure access is confirmed, Unmanned Aerial Vehicle 101 may facilitate transferring title or ownership of delivered assets from the merchant to a delivery agent, and then in turn to the end recipient.

Embodiments in accordance with the present disclosure include methods and apparatus that relate to the process of requesting and receiving assets such as a medical treatment kit and medical treatment kit supplies delivered by Unmanned Aerial Vehicles.

Embodiments may include in an Unmanned Aerial Vehicle a computing device and control infrastructure/server farm that keeps track of registered and unregistered pickup and delivery points, a Delivery Zones database, as well as perches where Unmanned Aerial Vehicles may be on standby to perform the transportation function with short notice. The computing device may be substantially any computer smart phone or communication device that may communicate with a Pickup and Delivery Database and/or server to request the pickup and delivery of a payload medical treatment kit.

Embodiments base a delivery location upon where the recipient may be at time of delivery. For example, a delivery location may be a moving vehicle or an individual who is publishing to a server an updated address location, in order for the Unmanned Aerial Vehicle to find the delivery point and estimate when it will arrive and the delivery point.

The process of requesting a pickup and delivery can include scheduling an available Unmanned Aerial Vehicle resources to be at the appropriate pickup point, together with an appropriate asset handling container that matches the physical profile of the goods assets or medical treatment kit to be delivered.

The pickup and delivery operations may include lining up Unmanned Aerial Vehicles or assets in aerial queues for the staging of thruster-equipped UAVs to deliver rapidly payloads during particular time frames at busy Delivery Zones.

The pickup and delivery process may include transferring of data that relate to other Unmanned Aerial Vehicle characteristics such as customs, intermodal, multi-modal, payment, and transport profile data. Transport profile data may include traditional black box data indicating the amount of G forces, temperature and humidity that took place during the flight.

A pickup and delivery process can include the delivery of assets such as a medical treatment kit as well as delivery of subcategory of assets (e.g., tools). The tools may be plugged into a standard housing (e.g., honeycomb type) for that particular tool type. This is similar to the delivery of mail/parcel in array of apartment mailboxes, personal mailboxes, remote control slide out drawers and business mail/parcel drop off points. These all may be managed via a mailbox Database that provides the registration of the mailboxes with the appropriate security access keys (e.g., physical and virtual/electronic keys with challenge response) and delivery profiles in precise latitude, longitude, and altitude data, plus valid time ranges.

Delivery also may include notification to a sender and a recipient of completion of the delivery, including GPS coordinates and imagery of what was physically delivered at the delivery location, as well as data representing the transfer of the asset at the particular Delivery Zone once it is closed and locked, thus guaranteed delivery and proof. The pickup and delivery also may take place between individual Unmanned Aerial Vehicles and a mailbox going back to a staging delivery vehicle which has, for example, all the mail on a given street or set of streets.

Vertical Delivery receptacles and Delivery Zones can include tubes, e.g., a vertically oriented honeycomb that then can direct merchandise assets into another facility by sliding down an air tube or slot for processing within another facility for final movement such as grocery stocking.

Unmanned Aerial Vehicle that pickup and deliver a medical treatment kit may include cameras that will authenticate a user space and voice pattern match with a database of local individuals who have been registered and verified. Verification may occur, for example via Governmental Identification Databases such as Passport or Province/State Driver's License, FaceBook, LinkedIn or some other large database of names and images.

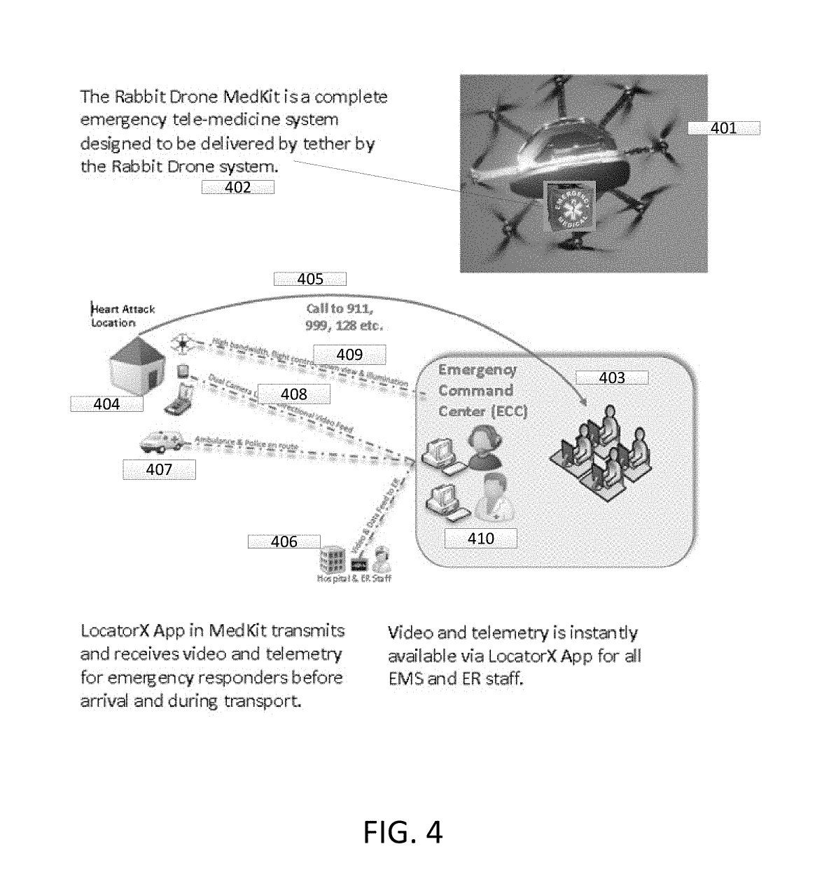

Referring now to FIG. 4, in some embodiments, a UAV 401 or other type of drone may be deployed to deliver a medical treatment kit 402 in response to a report of an emergency 405, or anticipation of an emergency. As illustrated in the example, a heart attack may be occurring at what will become the care giving location 404 is reported to an Emergency Command Center (ECC) 403. Other medical situations also are within the scope of embodiments of the present invention, and a heart attack is used for the sake of an exemplary illustration. In some embodiments, a report may be made via a call to 911, 99, 128 or other telephone number assigned to monitor emergencies. In other embodiments a report may be made via an app, a text message, an alert button, or other communications medium. ECC 403 may dispatch a UAV 401 and alerts medical staff, who will be in communication with a wireless communication device included in a medical treatment kit on the UAV 401.

UAV 401 during flight may switch into a video mode to both record events around during the UAV 401 travel and also to record and transmit imagery, environmental and location data events at a destination, which will typically be the site of an emergency.

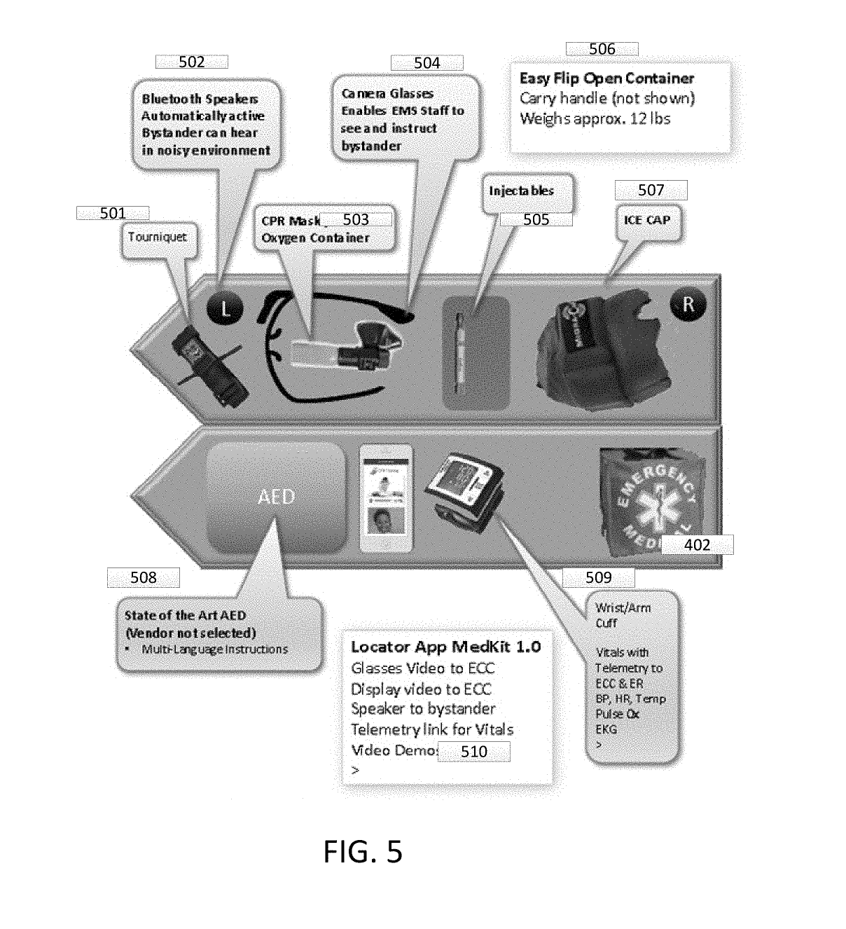

Relatively high bandwidth radio data links 406, 407, 408, 409 (e.g., 4G cellular data) allows for downward view and illumination and image capture. Camera Glasses 504 illustrated in FIG. 5, or any other type of headgear worn by a bystander, enable the ECC operator 410 to see what the bystander sees in real-time. Imagery captured by camera glasses 504 may be transmitted back to the ECC 403 and to the medical staff via radio data links 406, 407, 408, 409. The ECC operator 410 can instruct the bystander how to handle the components included in the medical treatment kit. As illustrated in FIG. 5, medical treatment kit 510 may be contained within an easy to open, but securely closable transport container such as container 506.

In some embodiments, a dual camera arrangement allows for live bi-directional video and audio feed, further enhancing the level of care that may be administered by allowing the bystander to know what the imagery looks like that they are sending to the ECC operator 410. Still further embodiments may include monitoring of the status and/or location of emergency response personnel, such as an ambulance or police or other first responder to the scene of the heart attack.

In some embodiments a cellular phone may act as a digital data gateway for various Bluetooth devices included in the medical treatment kit. A camera or other sensors included in the medical treatment kit may have incorporated within it a communication interface with the smartphone in order to transmit digital data via a cellular network.

In some embodiments, a virtual instruction screen may be worn by a first responder or other caregiver. The virtual instruction screen may include for example a headset, goggles, glasses or tablet that allow a caregiver to engage in hands-free communication and receive image based instruction while providing care to a patient. In some embodiments, the image based instruction will be controlled or provided by the ECC 403. In other embodiments the image based instruction will be provided by prerecorded video.

In some embodiments, a medical treatment kit also may include a set apart video capture device that allows an image capture device to be set up at a proximate distance, such as five feet from the site of care and capture the scene in greater scope. In some embodiments, the field of view for an image capture device may include and record emergency care, including all care givers and the patient. Individual caregivers may have supplemental image capture devices attached to themselves, such as in a headset, camera equipped glasses, a hat, a helmet, a body-worn device clipped onto clothing, and so forth. A multiplexer may receive and feed the imagery and telemetry data from sensors via cellular connection. In this manner, an ECC operator 410 may see what a technician, caregiver or bystander sees and hears.

In still another embodiment, electronic tracking (e.g., via electronic tags with unique identification codes) may help keep track of items have been removed from the medical treatment kit. The tracking of removal of the items may be transmitted to the ECC 403, and the ECC operator 410 then may comment back on whether a correct piece of equipment has been removed in order to perform care prescribed by the ECC operator 410.

The ECC operator 410 also may be made aware of an inventory of available items in the medical treatment kit via the electronic tag inventory. Using the available inventory of items in the medical treatment kit, the ECC operator 410 may prescribe care that is possible using the available items in the medical treatment kit.

In some embodiments, an image of an item a caregiver or bystander should remove from the medical treatment kit is transmitted to the caregiver or bystander in order to better facilitate a correct choice of a care giving device.

In another embodiment of the present invention, in order to address areas of substandard cellular coverage or interference, a care giving location 404 may relay up to a UAV 401 that hovers in area, acting as a traditional relay or repeater transmitter, proximate to the care giving location 404. UAV 401 receives wireless data from a device at the care giving location 404 and retransmits the data to the ECC 403. The retransmission may be at a higher power, such as a 5 watt transmission.

Referring now to FIG. 5, items that may be contained in an exemplary medical treatment kit 510 are illustrated. As illustrated, the kit 510 may be contained in an easy flip pen container 506, that may include a handle and be 12 lbs or less, the medical treatment kit may include, one or more of: a tourniquet 501, a Bluetooth audio and/or video device 502, a device comprising an oxygen container 503, multimedia headset or glasses 504, an injectable 505, an ice pack or cap 507; and AED 508, a wrist or arm cuff 509; and audio video devices with telemetry links.

In some embodiments, medical treatment kit 510 may be forward deployed where a need may be reasonably anticipated, e.g., near a stadium or other large gathering, or near known dangerous sites such as a rock climbing location. In some embodiments, the forward deployment may be on rooftop, with deployment via remote control and/or along a line of sight delivery path from the forward deployment location. Emergency workers or operators may monitor its usage.

In the above sections, detailed descriptions of embodiments of the invention have been given. The description of both preferred and alternative embodiments though through are exemplary only, and it is understood that to those skilled in the art that variations, modifications and alterations may be apparent. It is therefore to be understood that the exemplary embodiments do not limit the broadness of the embodiments of the underlying invention as defined by the claims.

1. A system to control placement of a payload carried by an unmanned aerial vehicle (UAV) at a delivery zone, the system comprising:
a payload coupled to the UAV;
a location determination device to determine a location of the UAV;
a drop line comprising a proximate end coupled to the UAV and a distal end for locating in a delivery zone;
a descent regulator to control a rate of descent of the payload from the UAV to the distal end of the drop line at the delivery zone via descent of the payload along the drop line; and
a release mechanism to release the payload from the drop line.

2. The system of claim 1, further comprising a position control unit to control a position of the UAV in a horizontal plane.

3. The system of claim 2, wherein the position control unit further controls one or both of:
an altitude of the UAV and a height of the UAV.

4. The system of claim 2, further comprising an altitude control unit to control one or both of an altitude of the UAV and a height of the UAV.

5. The system of claim 4, further comprising an operator interface to control navigation of the UAV based upon instructions transmitted to the position control unit.

6. The system of claim 1, further comprising a thruster coupled to the payload, the thruster to provide more accurate positioning of the payload in a lateral direction as compared to accuracy of lateral positioning enabled by the position control unit.

7. The system of claim 6, further comprising an operator interface to control positioning of the thruster.

8. The system of claim 1, wherein the delivery zone is described by image data and is recognized by the UAV by use of image recognition.

9. The system of claim 1, wherein the delivery zone comprises a mobile target.

10. The system of claim 1, wherein the delivery zone comprises a temporary target described by image data, and the temporary target is recognized by the UAV by use of image recognition.

11. The system of claim 1, wherein the delivery zone is marked by a radio transmitter.

12. The system of claim 1, wherein the payload comprises a medical treatment kit.

13. The system of claim 12, wherein the medical treatment kit comprises a heart defibrillator.

14. The system of claim 12, wherein the medical treatment kit comprises a voice communication link with an emergency command center.

15. A system providing precise placement of a payload carried by an unmanned aerial vehicle (UAV) to a delivery zone, the system comprising:
a mechanism to fixedly attach a payload to the UAV;
a location determination device to determine a location of the UAV;
a drop line comprising a proximate end coupled to the UAV and a distal end for positioning in a delivery zone;
a coupling device for slidingly attaching the payload to the drop line;
a descent regulator to control a rate of descent of the payload from the UAV to the delivery zone via the drop line;
a release mechanism to release the payload at the delivery zone;
an electronic control unit for controlling a location of the UAV;
a height controller for controlling a height of the UAV;
a thruster unit for controlling X and Y movement of the payload while the payload is suspended from the drop line;
a delivery zone determination unit for determining a location for delivery within a one meter accuracy; and
a delivery zone coupler for coupling the thruster unit to the delivery zone during delivery.

16. A method to control placement of an item carried by an unmanned aerial vehicle (UAV) at a delivery zone, the method comprising:
coupling a payload to the UAV;
determining a location of the UAV by use of a location determination device;
lowering the payload at the delivery zone, at a controlled rate of descent, by use of a drop line; and
releasing the payload at the delivery zone.

17. The method of claim 16, further comprising a step of, prior to releasing the payload, controlling a position of the payload separately from control of a position of the UAV.

18. The method of claim 16, further comprising a step of providing a voice communication link from the payload to an emergency command center.

19. The method of claim 16, further comprising a step of identifying the delivery zone by recognition of visible features.

20. The method of claim 16, further comprising a step of identifying the delivery zone by identification of non-visible features.
